(19) United States
(12) Patent Application Publication
(10) Pub. No.: US 2024/0401106 A1
(43) Pub. Date: Dec. 5, 2024

(54) Sample Preparation and Detection Methods Using Enzymatic Digestion

(71) Applicant: Neogen Corporation, Lansing, MI (US)

(72) Inventors: Preetha K. Biswas, Okemos, MI (US); Robert S. Donofrio, Williamston, MI (US); Ezzeddine Elmerhebi, Heysham (GB); Debra L. Foti, Fenton, MI (US); Fred Martinez, Montpellier (FR); Lei Zhang, Okemos, MI (US)

(21) Appl. No.: 18/695,069

(22) PCT Filed: Oct. 11, 2022
(86) PCT No.: PCT/US2022/077873

§ 371 Date: Mar. 25, 2024

Related U.S. Application Data
(60) Provisional application No. 63/254,228, filed on Oct. 11, 2021.

(51) Int. Cl. C12Q 1/22; C12Q 1/04; C12Q 1/34

(52) U.S. Cl. CPC C12Q 1/22; C12Q 1/04; C12Q 1/34

(57) ABSTRACT

Use of enzymes to digest food and environmental samples is described herein, which permit the separation, rapid capture and concentration of microorganisms or genomic template to achieve detection and/or identification.

### CROSS-REFERENCE TO RELATED APPLICATION

This application claims priority to and benefit of U.S. provisional application No. 63/254,228, filed Oct. 11, 2021, the entire contents of which are incorporated herein by reference.

### TECHNICAL FIELD

The present invention relates to the use of digestive enzymes to digest food and environmental samples to permit the rapid capture and concentration of microorganisms or genomic template to achieve detection and/or identification.

### BACKGROUND

In food and environmental microbiology testing, food-borne pathogens and spoilage organisms are usually tested to ensure the safety and quality of the food products. Sometimes the test portion specifications are larger than the sample size that can be tested by available detection methods. In addition, the microorganism numbers can be low, sparsely distributed, and unevenly distributed in the test portions, which adds to uncertainty of the sample size that needs to be delivered for effective detection by available methods. An enrichment step with long incubation periods is needed to allow the target microorganism to grow until the limit of detection of the detection method is reached. The enrichment step may require large volumes of growth media to be added to test samples adding to the challenges of streamlined sample handling and processing. Filtration may be used in place of enrichment to concentrate and isolate microorganisms, but only works for filterable test samples (i.e., non-viscous liquid samples). There is a need for new detection methods to provide faster, more efficient and reliable detection.

### SUMMARY

The present invention uses digestive enzymes to digest non-filterable samples. The digestion makes the sample suitable for concentration and capture by filtration or centrifugation, and can reduce or eliminate the need for an enrichment step or long sample incubation times to multiply the low level of microorganisms. In some embodiments, as an alternative to traditional filtration, handheld or miniaturized capture or filtration devices can be used. Various centrifugation protocols can also be utilized to capture microbial target(s). The enzymatic digestion also makes possible the release and detection of intracellular pathogen contamination, consequently enhancing assay sensitivity and leading to faster detection times.

In one aspect, provided herein is a method of preparing a food or environmental sample for detection of microbial contamination, the method comprising providing a food or environmental sample contaminated with a microbial contaminant; adding one or more digestion enzymes to the food or environmental sample; digesting the food or environmental sample using the one or more digestion enzymes to form a liquid digested sample; separating all or a portion of the liquid digested sample from remaining solid non-digested food or environmental sample material; and isolating the microbial contaminant in the liquid digested sample by passing the liquid digested sample through a membrane and capturing the microbial contaminant on the membrane. Alternatively, centrifugation and be used to sediment and capture microbial contaminants. In this instance the enzymatic treatment significantly reduces the solid sample mass captured during the centrifugation process, permitting better capture of the target microorganisms. Embodiments of this aspect may include one or more of the following optional features. In some embodiments, the one or more digestion enzymes comprises one or more digestion enzymes selected from the group consisting of pectinase, lactase, glucoamylase, acid amylase, saccharase, sucrase, cellulase, hemicellulase, beta-glucanase, xylanase, hydrolase, lyase, pectin methyl esterase, laccase, peptidase, protease, pepsin, trypsin, diastase, phospholipase, pullulanase, and lipase. In some embodiments, the one or more digestion enzymes comprises a hydrolase or lyase. In some embodiments, the food or environmental sample is a food sample. In some embodiments, the food or environmental sample is a solid-in-liquid suspension. In some embodiments, the solid-in-liquid suspension is a fruit juice. In some embodiments, the one or more digestion enzymes comprises pectinase and xylanase. In some embodiments, the one or more digestion enzymes comprises pectinase, hemicellulase, beta-glucanase, and xylanase. In some embodiments, the ratio of the one or more digestion enzymes to the solid-in-liquid suspension is from about 50:1 to about 200:1 by volume. In some embodiments, the food or environmental sample is a solid, and the method further comprises suspending the solid food or environmental sample in a liquid media. In some embodiments, the liquid media is an enrichment media, and the method further comprises enriching the microbial contaminant in the enrichment media at least partially concurrent with digesting the food or environmental sample. In some embodiments, the enrichment media is buffered peptone water. In some embodiments, the solid food or environmental sample is meat or other animal sourced foodstuff. In some embodiments the solid food or environmental sample is plant-based such as processed fruit or leafy greens/salad. In some embodiments, the concentration of the solid food or environmental sample suspended in the liquid media is from about 0.1 g/ml to about 2 g/ml (e.g., about 0.5 g/ml to about 2 g/ml). In some embodiments, digesting the food or environmental sample is performed for about 2 to about 8 hours. In some embodiments, digesting the food or environmental sample is performed for about 2 to about 4 hours. In some embodiments, digesting the food or environmental sample is performed at from about 30° C. to about 45° C. (e.g., from about 35° C. to about 40° C.). In some embodiments, the membrane for capturing the microbial contaminant has a pore size of from about 0.2 to about 0.5 μm (e.g., about 0.4 to about 0.5 μm). In some embodiments, the membrane for capturing the microbial contaminant is a cellulose nitrate membrane. In some embodiments the centrifugation utilizes 1 to 50 ml centrifuge tubes with up to 50 ml post digestion sample. In some embodiments the centrifugal force applied is from about 1000 to 8000 relative centrifugal force for about 1 to 10 minutes. In some embodiments, the microbial contaminant is bacteria, yeast, mold, fungus, virus, or parasite, or any combination thereof. In some embodiments, the microbial contaminant comprises one or more of Salmonella, Listeria, E. coli, Campylobacter, and Clostridium perfringens. In some embodiments, the food or environmental sample is a fruit juice and the one or more digestion enzymes comprises pectinase, hemicellulase, beta-glucanase, and xylanase. In some embodiments, the food or environmental sample is a fruit puree and the one or more digestion enzymes comprises pectinase, hemicellulase, beta-glucanase, and xylanase. In some embodiments, the method further comprises enriching the microbial contaminant for about 8 to about 24 hours at least partially concurrent with digesting the food or environmental sample. In some embodiments, the food or environmental sample is leafy greens and the one or more digestion enzymes comprises pectinase, hemicellulase, beta-glucanase, and xylanase.

In another aspect, provided herein is a method of detecting the presence of microbial contamination in a food or environmental sample, the method comprising preparing a food or environmental sample for detection of microbial contamination as described in any embodiment described herein; and detecting the presence of the microbial contaminant. Embodiments of this aspect may include one or more of the following optional features. In some embodiments, the method further comprises quantifying the microbial contamination. In some embodiments, the method further comprises plating the microbial contaminant onto one or more agar plates. In some embodiments, the method further comprises use of a rapid automated microbial detection platform, such as Soleris®. In some embodiments, the method further comprises performing a lateral flow assay, an enzyme-linked immunosorbent assay (ELISA), or a molecular method such as polymerase chain reaction (PCR) or isothermal amplification or sequencing and detection assay or a rapid automated microbial detection platform. In some embodiments, the total time to result is less than about 72 hours.

### BRIEF DESCRIPTION OF THE DRAWINGS

These and other features, aspects, and advantages of the present invention may be better understood when the following detailed description is read with reference to the accompanying drawing.

FIG. 1 is an exemplary workflow diagram for a method of detecting the presence of microbial contamination in a food or environmental sample according to the present invention.

FIG. 2 is a photograph showing 100 ml of orange juice post digestion demonstrating the separation of the non-digestible floccular mass and the filterable sample.

FIG. 3 is a plot of Soleris graph results of the seven duplicates of the digested and centrifuged samples from Example 2, showing positive detection of the spike.

FIG. 4 is a photograph showing the ability to digest leafy greens by suspending 20 g of lettuce in 100 ml of water and adding a mix of enzymes.

### DETAILED DESCRIPTION

In the following description, numerous specific details are given to provide a thorough understanding of the embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment,” “an embodiment,” or “embodiments” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless indicated otherwise, when a range of any type is disclosed or claimed, it is intended to disclose or claim individually each possible number that such a range could reasonably encompass, including any sub-ranges encompassed therein. Moreover, when a range of values is disclosed or claimed, which Applicants intend to reflect individually each possible number that such a range could reasonably encompass, Applicants also intend for the disclosure of a range to reflect, and be interchangeable with, disclosing any and all sub-ranges and combinations of sub-ranges encompassed therein.

The terms “comprises” and variations thereof do not have a limiting meaning where these terms appear in the description and claims.

As used herein, “a,” “an,” “the,” “at least one,” and “one or more” are used interchangeably. Thus, for example, a microorganism can be interpreted to mean “one or more” microorganisms.

The term “and/or” means one or all of the listed elements or a combination of any two or more of the listed elements.

The present invention relates to sample preparation methods for food or environmental samples. The methods prepare the sample for use in a detection method. A sample can be collected from a batch of food or from an environment (e.g., a surface) by known methods. In some embodiments, the food or environmental sample is a food sample. In some embodiments, the food sample is a solid-in-liquid suspension, e.g., a fruit juice. In some embodiments, the food sample is a solid, e.g., raw meat. Further non-limiting examples of food samples include raw or processed meat, raw or processed fruits or vegetables, salads, non-fluid dairy products (e.g., milk powder, cheese, butter, and ice cream), nuts, chocolate, spices, eggs, pet food, processed food, and syrups. Non-limiting examples of beverage sources include potable water, fruit or vegetable juices, milk, and fermented beverages. In some embodiments, the sample is a fruit juice (e.g., orange juice, pineapple juice, or mango juice). In some embodiments, the sample is a vegetable. In some embodiments, the sample is milk or milk powder. In some embodiments, the sample is raw meat. In some embodiments, the food or environmental sample is an environmental sample. In some embodiments, the environmental sample is selected from the group consisting of a collection device such as swab, sponge, cloth-process water, and a primary production sample (that can contain contamination from food, dust, soil or feces).

In some embodiments, the food or environmental sample is non-filterable, i.e., microbial contaminants cannot be effectively separated from the sample by use of a filter. Using the present methods, the food or environmental sample is made filterable such that the microbial contaminants can be effectively separated from the sample by use of a filter. In some embodiments, the food or environmental sample has a mass of from about 25 g to about 375 g (e.g., from about 125 to about 375 g). Digestion, followed by filtration, allows for the processing of larger samples due to the efficiency of concentration (e.g., filtration).

The food or environmental sample is contaminated with a microbial contaminant. In some embodiments, the microbial contaminant is bacteria, yeast, mold, fungus, virus, or parasite, or any combination thereof. In some embodiments, the microbial contaminant comprises one or more of Salmonella, Listeria, E. coli, Campylobacter, and Clostridium perfringens.

Upon collection of a food or environmental sample, the sample undergoes enzymatic digestion. For solid-in-liquid suspension samples, one or more digestive enzymes may be added directly to the sample, without prior processing (i.e., undiluted). Alternatively, the suspension may be diluted with a liquid in conjunction with adding the digestive enzymes. In some embodiments, the liquid is a sterile diluent, e.g., phosphate buffered saline (PBS). In some embodiments, the liquid is an enrichment media, e.g., an enrichment broth. In some embodiments the enrichment media is buffered peptone water. The suspension may be diluted with the liquid in a dilution ratio of from about 1:1 to about 1:10 (e.g., 1:2, 1:3, or 1:4).

For solid samples, a liquid is added to the sample, and forms a mixture together with the digestive enzymes. In some embodiments, the liquid is a sterile diluent, e.g., PBS. In some embodiments, the liquid is an enrichment media, e.g., an enrichment broth. In some embodiments the enrichment media is buffered peptone water. In some embodiments, the concentration of the solid food or environmental sample suspended in the liquid media is from about 0.1 to about 2 g/ml (e.g., about 0.5 to about 2 g/ml).

The digestive enzyme digests the food or environmental sample. Digestion helps to release the microbial contaminants from the matrix of the food or environmental sample, so that the contaminants can be isolated by filtration. Digestion also breaks down the food or environmental sample (e.g., liquefying the sample) so that the sample can be passed through a filter that will capture the microbial contaminants and/or digestion reduces the solid sample mass captured during the centrifugation process, permitting better capture of the target microorganisms.

The digestive enzymes digest the food or environmental sample to form a digested sample. Digestion breaks down the food or environmental sample. In some embodiments, the non-digested sample is a non-filterable sample and, after digestion, the digested sample is filterable. Digestion also releases microbial contaminants from the food or environmental sample. Digestion times are short compared to times required for many process steps in convention sample preparation, which may take many hours or days. In some embodiments, digestion is performed in less than 24 hours, preferably less than 16 hours, more preferably less than 12 hours. For example, digestion is performed for from about 1 to about 16 hours, from about 4 to about 16 hours, from about 8 to about 16 hours, from about 1 to about 8 hours, from about 2 to about 8 hours, from about 4 to about 8 hours, from about 1 to about 4 hours, from about 2 to about 4 hours, or from about 1 to about 2 hours. In some embodiments, the sample is completely digested. In some embodiments, the sample is partially digested (e.g., about 80% or more). Digestion may be performed at a sample temperature of about 30 to about 45° C., e.g., about 35 to about 40° C., or about 37° C.

In some embodiments the one or more digestive enzymes comprise one or more digestive enzymes selected from the group consisting of pectinase, lactase, glucoamylase, acid amylase, saccharase, sucrase, cellulase, hemicellulase, beta-glucanase, xylanase, hydrolase, lyase, pectin methyl esterase, laccase, peptidase, protease, pepsin, trypsin, diastase, phospholipase, pullulanase, and lipase. In some embodiments, the one or more digestive enzymes are selected from the group consisting of pectinase, lactase, glucoamylase, acid amylase, saccharase, sucrase, cellulase, hemicellulase, beta-glucanase, xylanase, hydrolase, lyase, pectin methyl esterase, laccase, peptidase, protease, pepsin, trypsin, diastase, phospholipase, pullulanase, and lipase, or any combination thereof. In some embodiments, the one or more digestion enzymes comprises hydrolase or lyase. In some embodiments, the one or more digestive enzymes comprise or consist of pectinase and xylanase. In particular, pectinase and xylanase may be used as the digestive enzymes when the sample is a fruit juice (e.g., orange juice). In some embodiments, the one or more digestive enzymes comprise or consist of pectinase, hemicellulase, beta-glucanase, and xylanase. Foodborne pathogens will survive the enzymatic digestion, at least to the extent necessary for detection. In some embodiments, the concentration of digestive enzymes added to the food or environmental sample is about 1% to about 15% by weight of the sample, e.g., from about 1% to about 5%, from about 1% to about 3%, from about 5% to about 15%, or from about 8% to about 12%.

In some embodiments, the method of preparing a food or environmental sample for detection of microbial contamination does not include enrichment. Eliminating the need for enrichment greatly decreases the time needed for sample preparation prior to detection compared to conventional methods. In other embodiments, the method of preparing a food or environmental sample for detection of microbial contamination includes a short enrichment step. In embodiments where enrichment is used, enrichment time is shorter than conventional sample preparation methods, which generally include enrichment times of 16 to 48 hours. For example, enrichment times in the present methods may be from about 1 to about 8 hours, e.g., from about 1 to about 4 hours, or from about 1 to about 2 hours, or from about 2 to about 4 hours. In some embodiments, the sample is enriched to increase the number and concentration of microbial contaminant material in the sample, to aid in detection of microbial contaminant in the sample. Enrichment occurs at least partially concurrently with digestion of the sample. Enrichment media (e.g., enrichment broth) is used as a diluent for the sample and forms a mixture together with the sample and the digestive enzymes. Digestion and enrichment thus occur at least partially concurrently with the digestive enzymes digesting the sample and releasing microbial contaminants from the food or environmental sample into the enrichment media. To aid enrichment, the sample may be kept at a suitable enrichment temperature, e.g., about 30-45 or about 35-40° C., e.g., about 37° C. Without being bound by theory, the enzymatic digestion is also understood to aid the enrichment process by reducing inhibitory factors, creating short chain compounds available for microbial digestion and improving microbial kinetics by altering the consistency/viscosity.

After digestion of the sample by the digestive enzymes, the present methods comprise isolation of the microbial contaminants. Isolation involves separating the microbial contaminants from all or a portion of the other components of the sample. In some embodiments, an initial filtration step is employed to remove larger pieces of undigested food or environmental material in the sample. A suitable filter may be used to allow the liquid digested sample to flow through while filtering out the undigested solid material. In some embodiments, a subsample is collected from the digested material. For example, a pipette may be used to collect a subsample of the digested material to isolate the digested material from the non-digested material. Collection of a subsample that is substantially free of non-digested material may be made possible by allowing solid non-digested material to settle after digestion.

Further isolation is employed to prepare for detection of microbial contaminants. Specifically, the liquid digested sample (or subsample) may be passed through a filter that captures the microbial contaminants. Suitable filters include analytical filters. In some embodiments, the filter comprises a membrane having a pore size of from about 0.2 to about 0.5 μm (e.g., from about 0.4 to about 0.5 μm, e.g., about 0.45 μm). In some embodiments, the filter comprises a cellulose nitrate membrane. Syringe filter devices may also be used.

Alternative to the second filtration process, further isolation may be employed to prepare for detection of microbial contaminants via a centrifugation method. Specifically, the liquid digested sample (or subsample) may be aliquoted into a centrifuge tube and spun for 5 minutes at 4000 RPM (2742 RCF), the centrifugal force applied is from about 1000 to 8000 relative centrifugal force for about 1 to 10 minutes. The supernatant is then discarded, and remaining pellet with the concentrated microorganisms is used for subsequent analysis steps. The centrifugation for a pre-digested sample will result in a smaller size pellet of concentrated microorganisms removing interfering large particles and large volumes of liquid which dilutes the organism of interest. This improves the chance of identifying low levels of microorganisms spread in large volumes of digested liquified sample.

By utilizing digestion of the food or environmental sample, optionally followed by removal of nondigested pieces, the non-filterable sample becomes a filterable sample that can be filtered to capture the microbial contaminants without clogging the filter. The digestion process also releases the microbial contaminants from the sample, making them available for capture from the liquid sample, and ultimately available for detection.

Alternatively, other isolation methods can be employed to isolate the microbial contaminant or genomic template thereof from the digested food or environmental sample. For example, separation by immunomagnetic beads can be used to isolate the microbial contaminant or genomic template thereof. Various cellular or DNA/RNA extraction devices or techniques may also be used; for example, PCR or isothermal molecular detection.

Upon isolation of the microbial contaminant from the sample, the microbial contaminants can be detected. In some embodiments, the method further comprises quantifying the microbial contamination. In some embodiments, detection comprises plating the microbial contaminant onto one or more agar plates or the use of a rapid automated microbial detection platforms. In some embodiments, detection comprises use of a rapid automated microbial detection platform, such as Soleris®. In some embodiments, detection comprises performing a lateral flow assay, an enzyme-linked immunosorbent assay (ELISA), or a polymerase chain reaction (PCR) amplification and detection assay. In some embodiments, detection comprises a culture media-based detection or a molecular system. In some embodiments, the method employs a detection method selected from the group consisting of PCR, DNA sequencing, molecular probes, isothermal detection, lateral flow assay, ELISA, ELFA, petri culture, and detection broth culture.

In some embodiments, the method further comprises additional sample preparation to prepare the isolated microbial contaminant for a detection method. For example, the microbial contaminant may be lysed to release cellular components (DNA, RNA, etc.) for detection methods based on detection of cellular components.

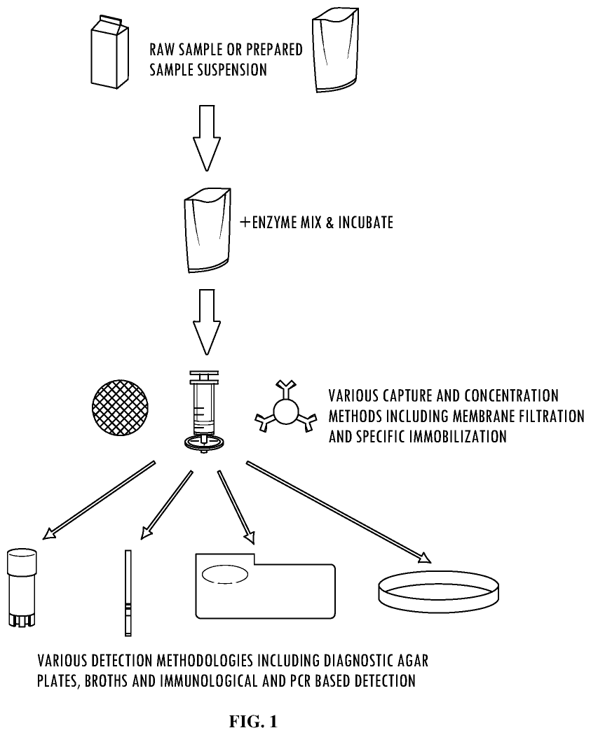

In some embodiments, the total time to result (i.e., detection of microbial contaminant) is from about 2 to about 72 hours, e.g., from about 24 to about 72 hours, from about 48 to about 72 hours, from about 24 to about 64 hours, or from about 48 to about 64 hours. In some embodiments, the total time to result is from about 2 to about 48 hours, e.g., about 24-48 hours, about 2-24 hours, about 2-12 hours, about 2-8 hours, about 4-24 hours, about 4-12 hours, about 4-8 hours, about 8-24 hours, or about 8-12 hours. A sample workflow diagram is shown in FIG. 1.

Aspects of the invention also include kits with components used in the methods described herein. Kits may include one or more of: a device for collecting a food or environmental sample, one or more digestive enzymes, a sterile diluent, enrichment media, a container for digestion and optionally for enrichment, an incubator, filter(s), centrifuge, and detection product(s) (e.g., agar plate(s)). Kits may also include instructions for performing a method as described herein.

### EXAMPLES

### Example 1-Rapid Spoilage Microorganism Detection Protocol for Unfilterable Fruit Juice

The performance of the enzymatic digestion protocol was evaluated following ISO 16140-2 using range of unfilterable juice drinks including orange juice from concentrate.

The traditional method tests for acidic tolerant spoilage microorganisms by enriching a 10 ml sample in 90 ml of malt extract broth at 28° C. to 32° C. for 72±2 hours. This is followed by 1 ml subculture in orange serum agar as a pour plate. The plate is incubated for 3 days in an incubator set at 28° C. to 32° C. Presence of colonies after incubation indicates presumptive spoilage in the original sample. The entire detection protocol takes at least 6 days to yield a result. Pre-enrichment is required to detect presence of spoilage in the whole sample, rather than a smaller portion of it.

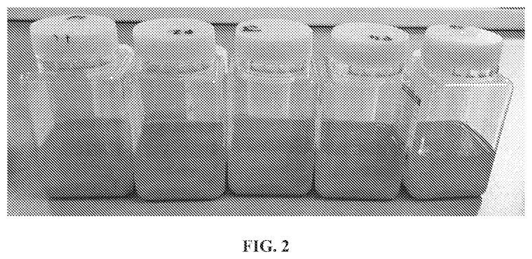

Enzymatic digestion of the unfilterable juices was performed using 2% mix of pectinases, hemicellulases, beta-glucanases/xylanases in 100 ml sample volumes. The stabilised liquid enzymes were added directly to the various different juices and statically incubated for 2 hours at 37° C. After incubation, in most cases, there was a fraction of undigested material that drops to the bottom of the container which presents as a floccular mass. The remainder is translucent filterable liquid as displayed in FIG. 2, which shows 100 ml of orange juice post digestion demonstrating the separation of the non-digestible floccular mass and the filterable sample.

In samples with a lower juice content, the enzymatic treatment can result in almost complete digestion but in all cases only 50 ml of sample was taken to capture using membrane filtration. This was done by transferring to an analytical filter funnel with a 0.45 μm membrane filter. A vacuum is applied, and the sample is filtered in <5 minutes. Any microorganisms present are trapped on the membrane, which is transferred to the detection methodology (either agar or Soleris vial). In this instance the NF-OSB vial was tested using enzymatic digestion/membrane filtration against the reference method using pre-enrichment and plating. After digestion the membrane filter is aseptically removed and placed in an NF-OSB vial, which is incubated for 30° C. for 48 hours in the Soleris system. A relative limit of detection study was carried out following the rules of ISO 16140-2:2016. Pasteurized orange juice was used as the matrix and Candida albicans NCPF 3255 was used as the contaminants. The study used 5 replicates of blank (negative) samples, 20 replicates of a low spike of 0.7 CFU/ml and 5 replicates of a high spike of 1.5 CFU/ml. The samples were paired as 200 ml in total was used for each test, 100 ml was tested by pre-enrichment and 100 ml by the enzymatic digestion and Soleris detection method. The results showed that both methods yielded 0/5 detections for the blank samples as well as 5/5 detections. For the low spike 10/20 detections were observed for the reference method and 9/20 were observed for the alternative method using enzymatic digestion. With only one deviation at the low spike out of 20, there is no significant difference between the reference and alternative method. The enzymatic digestion protocol achieved a relative limit of detection of around 1 CFU per 100 ml sample tested, whilst not requiring 3 day pre-incubation because of the ability to digest and filter the sample.

### Example 2: Example Protocol for Detection of Yeast and Mold in Fruit Purees Using Centrifugation

Following the rules of ISO 16140-3:2021, a verification was carried out using an enzymatic digestion protocol for strawberry fruit puree. In duplicate sets of 7 samples, a 100 g of fruit puree product spiked with 5 CFU of Candida albicans ATCC 10231, were weighed into a stomacher bag and 300 mL of Sabouraud dextrose broth was added. A 10% mix of pectinases, hemicellulases, beta-glucanases/xylanases was added to the suspension. Each sample was pre-enriched for 16 hours at 25° C.+/−1° C. The samples were removed from the incubator carefully and 50 mL was removed from the digested fraction using a serological pipette and added to a centrifuge tube. Each sample was centrifuged for 5 minutes at 4000 RPM (2742 RCF). The supernatant was poured off, taking care to retain the pellet. The pellet was resuspended in 5 mL of broth from a DYM-109C yeast and mold vial and then vortexed, the broth was then reintroduced to the DYM-109C vial. The vial was tested using the standard Soleris parameters for this vial utilizing a 48-hour test run.

The reference method consisted of 7 replicates of 100 g of strawberry fruit puree samples spiked with 5 CFU of Candida albicans ATCC 1023, which were then weighed into a stomacher bag and 400 mL of Sabouraud dextrose broth added. Each sample was pre-enriched for 48 hours at 25° C.+/−1° C. The samples were removed from the incubator and 0.5 mL was pipetted into duplicate yeast extract glucose chloramphenicol (YGC) plates. The plates were incubated for 3 days at 25° C.+/−1° C.

The following results show the performance of the alternative method (using enzymatic digestion) in a total time of 64 hours verses the plate results of the reference method with a total time of 5 days.

TABLE 1
Soleris and Plate Count Results

| | Soleris Detection Time | Plate Results YGC |
|---|---|---|
| Sample | (Hours) | (Presence/Absence) |
| 1 | 15.6 | Presence |
| 2 | 16.2 | Presence |
| 3 | 17.0 | Presence |
| 4 | 17.8 | Presence |
| 5 | 19.0 | Presence |
| 6 | 17.2 | Presence |
| 7 | 17.6 | Presence |
| Blank | Not Detected | |

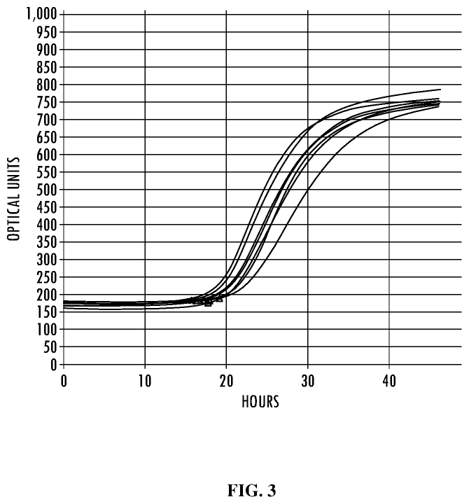

FIG. 3 shows Soleris graph results of all 7 duplicates of the digested and centrifuged samples showing positive detection of the spike.

The results show concordance with the spike detected in all tests. The enzymatic digestion protocol permits to avoid the 2 day incubation by breaking the sample down enough so that the resulting centrifugal pellet (containing any cells) is small enough to introduce into a standard detection method.

### Example 3: Example of Digestion with a Solid Non-Fruit Based Matrix

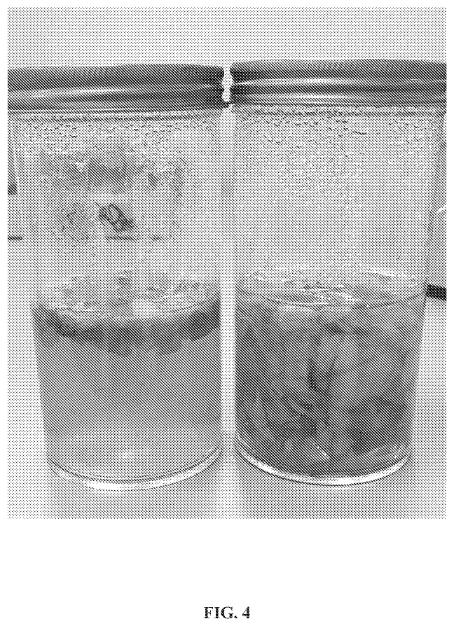

The enzyme mixtures can be applied to a wide range of matrices and by creating sample suspensions, solid samples to be analyzed. FIG. 4 shows the ability to digest leafy greens by suspending 20 g of lettuce in 100 ml of water and adding a mix of pectinases, hemicellulases, and beta-glucanases/xylanases.

FIG. 4 is an example of the digestion of leafy greens. Both samples are 20 g of lettuce suspended in 100 ml of water and incubated for 2 hours at 37° C. in a static aerobic incubator. The sample on the left has been supplemented with the enzyme mixture whereas the same on the right has not.

Digestion of the sample means that the resulting liquid can be filtered to capture any microbial contaminants (including pathogens). Whereas without the digestion the 20 g of lettuce would require pre-enrichment in broth for at least 24 hours to reliably be able to detect potential contaminants.

### Example 4: Example Protocol for Raw Meat

375 ml of an enzyme mix+Buffered Peptone Water is used to digest and enrich up to 375 g of matrix (minced beef meat) for 4-8 hours at 37° C. in a standard air incubator. Any samples that contain large “bits” must be filtered using a suitable blending bag with a filter membrane. Following the digestion plus short enrichment step, 100 ml is removed using a serological pipette and electronic pipettor. The sub sample is transferred to a single use analytical filter funnel with a 0.45 μm membrane filter. A vacuum is applied, and the sample should pass through in <5 minutes. The filter paper is then aseptically extracted and folded in order to insert onto Salmonella chromogenic agar plates (CASE), a rapid diagnostic method. The plates are incubated 18-24 hours before reading. As noted above, methodologies for testing raw meat require long time for sample pre-enrichment before testing with a rapid diagnostic method such as Salmonella Chromogenic agar, which can be shortened with use of the present enzyme digestion method.

1. A method of preparing a food or environmental sample for detection of microbial contamination, the method comprising
providing a food or environmental sample contaminated with a microbial contaminant;
adding one or more digestion enzymes to the food or environmental sample;
digesting the food or environmental sample using the one or more digested enzymes to form a liquid digested sample;
separating all or a portion of the liquid digested sample from remaining solid non-digested food or environmental sample material; and one or both of the following steps:
(a) isolating the microbial contaminant in the liquid digested sample by passing the liquid digested sample through a membrane and capturing the microbial contaminant on the membrane, or (b) isolating the microbial contaminant in the liquid digested sample by centrifugation.

2. The method of claim 1, wherein the one or more digestion enzymes comprises one or more digestion enzymes selected from the group consisting of pectinase, lactase, glucoamylase, acid amylase, saccharase, sucrase, cellulase, hemicellulase, beta-glucanase, xylanase, hydrolase, lyase, pectin methyl esterase, laccase, peptidase, protease, pepsin, trypsin, diastase, phospholipase, pullulanase, and lipase.

3. The method of claim 1, wherein the food or environmental sample is a food sample.

4. The method of claim 1, wherein the food or environmental sample is an environmental collection device.

5. The method of claim 1, wherein the food or environmental sample is a solid-in-liquid suspension.

6. The method of claim 5, wherein the solid-in-liquid suspension is a fruit juice.

7. The method of claim 5, wherein the one or more digestion enzymes comprises pectinase and xylanase.

8. The method of claim 5, wherein the ratio of the one or more digestion enzymes to the solid-in-liquid suspension is from about 50:1 to about 200:1 by volume.

9. The method of claim 1, wherein the food or environmental sample is a solid; the method further comprising suspending the solid food or environmental sample in a liquid.

10. The method of claim 9, wherein the liquid is an enrichment media; the method further comprising enriching the microbial contaminant in the enrichment media at least partially concurrent with digesting the food or environmental sample.

11. The method of claim 10, wherein the enrichment media is buffered peptone water.

12. The method of claim 9, wherein the solid food or environmental sample is fruit, leafy greens, or meat.

13. The method of claim 9, wherein the concentration of the solid food or environmental sample suspended in the liquid is from about 0.1 g/ml to about 2 g/ml.

14. The method of claim 1, wherein digesting the food or environmental sample is performed for about 2 to about 8 hours.

15. The method of claim 14, wherein digesting the food or environmental sample is performed for about 2 to about 4 hours.

16. The method of claim 14, wherein digesting the food or environmental sample is performed at from about 30 to about 45° C.

17. The method of claim 1, wherein the membrane for capturing the microbial contaminant has a pore size of from about 0.2 to about 0.5 μm.

18. The method of claim 17, wherein the membrane for capturing the microbial contaminant is a cellulose nitrate membrane.

19. The method of claim 1, comprising centrifuging the liquid digested sample by applying a centrifugal force from about 1000 to 8000 RCF for about 1 to 10 minutes.

20. The method of claim 1, wherein the microbial contaminant is bacteria, yeast, mold, fungus, virus, or parasite, or any combination thereof.

21. The method of claim 20, wherein the microbial contaminant comprises one or more of Salmonella, Listeria, E. coli, Campylobacter, and Clostridium perfringens.

22. The method of claim 1, wherein the one or more digestion enzymes comprises pectinase, hemicellulase, beta-glucanase, and xylanase.

23. The method of claim 1, wherein the food or environmental sample is a fruit juice and the one or more digestion enzymes comprises pectinase, hemicellulase, beta-glucanase, and xylanase.

24. The method of claim 1, wherein the food or environmental sample is a fruit puree and the one or more digestion enzymes comprises pectinase, hemicellulase, beta-glucanase, and xylanase.

25. The method of claim 24, further comprising enriching the microbial contaminant for about 8 to about 24 hours at least partially concurrent with digesting the food or environmental sample.

26. The method of claim 1, wherein the food or environmental sample is leafy greens and the one or more digestion enzymes comprises pectinase, hemicellulase, beta-glucanase, and xylanase.

27. A method of detecting the presence of microbial contamination in a food or environmental sample, the method comprising
preparing a food or environmental sample for detection of microbial contamination according to the method of claim 1; and
detecting the presence of the microbial contaminant.

28. The method of claim 27, further comprising quantifying the microbial contamination.

29. The method of claim 27, wherein detecting the presence of the microbial contaminant comprises plating the microbial contaminant onto one or more agar plates.

30. The method of claim 27, wherein detecting the presence of the microbial contaminant comprises performing a lateral flow assay, an enzyme-linked immunosorbent assay (ELISA), a polymerase chain reaction (PCR) amplification and detection assay, or a rapid automated microbial detection platform.

31. The method of claim 27, wherein the total time to result is less than about 72 hours.
